(19) United States
(12) Patent Application Publication
(10) Pub. No.: US 2026/0347954 A1
(43) Pub. Date: Nov. 5, 2026

(54) SULFUR-CONTAINING PROBE FOR COLORIMETRIC DETECTION, AND METHOD FOR ACCELERATING ENZYMATIC ACTIVITY THROUGH SULFUR INTRODUCTION INTO NUCLEIC ACIDS

(71) Applicant: KOREA UNIVERSITY RESEARCH AND BUSINESS FOUNDATION, Seoul (KR)

(72) Inventors: Cheul Hee JUNG, Seoul (KR); Hyun SHIN, Anyang-si (KR)

(21) Appl. No.: 19/119,517

(22) PCT Filed: Oct. 12, 2023
(86) PCT No.: PCT/KR2023/015709

§ 371 Date: Apr. 9, 2025

(30) Foreign Application Priority Data
Oct. 14, 2022 (KR) 10-2022-0132471
Oct. 12, 2023 (KR) 10-2023-0135804

(51) Int. Cl. C12Q 1/70

(52) U.S. Cl. CPC C12Q 1/701

(57) ABSTRACT

The present disclosure relates to a sulfur-containing probe for colorimetric detection, and a method for accelerating enzymatic activity through sulfur introduction into nucleic acids. Particularly, the present disclosure relates to: a sulfur-containing probe for colorimetric detection, wherein the probe has the form of a DNA oligonucleotide in which nucleobases are continuously connected, the continuous connection of nucleobases is characterized in that any one or more selected from adenine (A), guanine (G), cytosine (C), and thymine (T) are randomly connected and the nucleotide phosphate backbone of the DNA oligonucleotide is modified with one or more phosphorothioates so as to include one or more sulfur atoms; a composition containing the probe for colorimetric detection; a kit for colorimetric detection; a colorimetric detection method; and a method for preparing the sulfur-containing probe for colorimetric detection, having the effect of enhancing enzymatic activity mediated by colorimetric detection.

### TECHNICAL FIELD

The present disclosure relates to a sulfur-containing probe for colorimetric detection and a method for accelerating enzymatic activity through sulfur introduction into nucleic acids.

### BACKGROUND ART

The COVID-19 pandemic, caused by the SARS-COV-2 virus, continues to spread globally and has not yet been fully controlled. In response to the pandemic, among various methods for detecting the virus, a representative method is fluorescence-based detection such as a quantitative PCR using a fluorescence-labeled probe. This method is very sensitive and specific, but has several limitations. The method requires expensive equipment and trained personnel, and the detection process is time-consuming. These limitations are a significant constraint, particularly in the current pandemic situation where the importance of the spread and prevention of infectious diseases has been emphasized, and thus there is a need for the development of new methods that are more rapid, user-friendly, and cost-effective for on-site diagnosis.

On the other hand, a colorimetric diagnosis method has an advantage of being able to interpret detection results without expensive equipment or skilled personnel for detection, and being suitable for repetitive and large-scale group screening compared to fluorescence analysis in a diagnostic cost-effective manner. In addition, the method is easy to handle and may simply and rapidly confirm results, and thus is suitable for application to on-site-based nucleic acid diagnosis.

However, DNAzyme used for such colorimetric diagnosis is a type of DNA oligonucleotide that may carry out specific chemical reactions, particularly known for its ability to mimic the activity of peroxidase when used in a G-quadruplex/hemin DNAzyme system. This system is used in diagnostic methods capable of producing colorimetric signals. G-quadruplex has a structure formed by four strands of guanine-rich oligonucleotides that are linked to each other by Hoogsteen base pairing to form a stack of G-quartets. When hemin is added to this structure, hydrogen peroxide-mediated oxidation may occur, which leads to the oxidation of substrates such as ABTS, luminol, OPD, and TMB, enabling colorimetric diagnosis. However, this method has the limitation of requiring the presence of the G-quadruplex structure for colorimetric diagnosis, and thus it is difficult to design sequence-specific probes using a G-quadruplex structure, and there is a problem in that it is impossible to distinguish false positives from non-specific amplification.

Therefore, there is a need to develop a new colorimetric diagnosis method that may address these problems and is more convenient and effective.

### DISCLOSURE

### Technical Problem

Therefore, the present inventors designed a new probe for colorimetric detection in which sulfur atoms, which are not present in nucleic acids but affect enzymatic activity, are introduced into nucleic acids through phosphorothioate bonds (PS) with a new concept DNAzyme that is not restricted by a sequence, so as to improve the disadvantages of existing colorimetric diagnosis, and confirmed that when the probe is used, enzymatic activity is accelerated, enabling more rapid and accurate colorimetric diagnosis, and thus completed the present disclosure.

Therefore, the present disclosure is directed to providing a sulfur-containing probe for colorimetric detection, in which the probe has the form of a DNA oligonucleotide in which nucleobases are continuously connected, and the continuous connection of nucleobases is characterized in that any one or more selected from adenine (A), guanine (G), cytosine (C), and thymine (T) are randomly connected, and a nucleotide phosphate backbone of the DNA oligonucleotide is modified with one or more phosphorothioates so as to include one or more sulfur atoms.

The present disclosure is further directed to providing a composition for colorimetric detection including the sulfur-containing probe for colorimetric detection of the present disclosure.

The present disclosure is further directed to providing a kit for colorimetric detection including the composition for colorimetric detection of the present disclosure.

The present disclosure is further directed to providing a colorimetric detection method using the sulfur-containing probe for colorimetric detection of the present disclosure.

The present disclosure is further directed to providing a method for preparing a sulfur-containing probe for colorimetric detection, having an effect of enhancing enzymatic activity mediated by a colorimetric reaction, the method including selecting a target gene to be detected, designing an oligonucleotide sequence complementary to a nucleic acid sequence of the target gene, and modifying a nucleotide phosphate backbone in the complementary oligonucleotide with one or more phosphorothioates, wherein the complementary oligonucleotide has a length of 10 to 30 mer, and the phosphorothioate modification is performed in a region in which at least one of nucleobases present on either side of the modification is adenine (A).

### Technical Solution

Therefore, an aspect of the present disclosure provides a sulfur-containing probe for colorimetric detection, in which the probe has the form of a DNA oligonucleotide in which nucleobases are continuously connected, and the continuous connection of nucleobases is characterized in that any one or more selected from adenine (A), guanine (G), cytosine (C), and thymine (T) are randomly connected and a nucleotide phosphate backbone of the DNA oligonucleotide is modified with one or more phosphorothioates so as to include one or more sulfur atoms.

In one embodiment of the present disclosure, the sulfur-containing probe for colorimetric detection may have the form of a DNA oligonucleotide in which 10 to 30 nucleobases are continuously connected.

In one embodiment of the present disclosure, the sulfur-containing probe for colorimetric detection may enhance enzymatic activity mediated by the colorimetric reaction.

In one embodiment of the present disclosure, when the phosphate backbone of the DNA oligonucleotide is modified with phosphorothioates, at least one of the nucleobases present on either side of the modification may be adenine (A).

In one embodiment of the present disclosure, the DNA oligonucleotide including one or more sulfur atoms may be a single-stranded DNA oligonucleotide.

Further, another aspect of the present disclosure provides a composition for colorimetric detection including the sulfur-containing probe for colorimetric detection of the present disclosure.

In one embodiment of the present disclosure, the composition may further include a colorimetric reagent.

In one embodiment of the present disclosure, the colorimetric reagent may include at least one selected from the group consisting of 2,2′-azino-bis(3-ethylbenzothiazoline-6-sulfonic acid) (ABTS), o-phenylenediamine dihydrochloride (OPD), diaminobenzidine (DAB), 3-amino-9-ethylcarbazole (AEC), 3,3′,5,5′-tetramethylbenzidine (TMB), AmplexRed, and Homovanillic acid, and at least one peroxide.

In one embodiment of the present disclosure, the composition may further include hemin.

In addition, in one embodiment of the present disclosure, the composition may further include hydrogen peroxide (H₂O₂).

Yet another aspect of the present disclosure provides a kit for colorimetric detection including the composition for colorimetric detection of the present disclosure.

Still another aspect of the present disclosure provides a colorimetric detection method using the sulfur-containing probe for colorimetric detection of the present disclosure.

Still another aspect of the present disclosure provides a method for preparing a sulfur-containing probe for colorimetric detection, having an effect of enhancing enzymatic activity mediated by a colorimetric reaction, the method including selecting a target gene to be detected, designing an oligonucleotide sequence complementary to a nucleic acid sequence of the target gene, and modifying a nucleotide phosphate backbone in the complementary oligonucleotide with one or more phosphorothioates, wherein the complementary oligonucleotide has a length of 10 to 30 mer, and the phosphorothioate modification is performed in a region in which at least one of nucleobases present on either side of the modification is adenine (A).

### Advantageous Effects

According to the present disclosure, it is possible to solve the problem of colorimetric diagnosis having sequence limitations by introducing sulfur into nucleic acids. That is, a new concept of DNAzyme exhibiting peroxidase activity by introducing sulfur, an element that does not exist in nucleic acids according to the present disclosure, has an effect of expanding the applicability of colorimetric diagnostic technology by confirming positive and negative detection results depending on a DNA form. In addition, unlike existing DNAzymes that essentially require a structure, the DNAzyme of the present disclosure has a highly general effect that can be widely applied to various sequences without a specific structure for colorimetry and additional modification, and also has an advantages of being inexpensive and easy to be introduced by using phosphorothioate modification that makes it easy to introduce sulfur into nucleic acids. In addition, while sulfur included in the monomer form has no colorimetric activity, sulfur modified single stranded DNA of a sufficient length can interact with hemin to generate peroxidase-like activity, and when complementary sequences are hybridized, bases participating in the reaction are limited to react with a substrate by hydrogen bonding to confirm a colorimetric quenching effect, and thus, have an effect of enabling simple, rapid, and accurate colorimetric detection and diagnosis compared to conventional methods.

### DESCRIPTION OF DRAWINGS

FIG. 1 shows an effect of introducing sulfur into nucleic acid, showing A) connection structures between phosphodiester bond (PO) and phosphorothioate bond (PS) nucleotides, respectively, B) a schematic diagram of a difference in enzymatic activity according to the presence or absence of PS modification, confirming that the enzymatic activity increases when PS modification is introduced into ssDNA, C) a result of colorimetric analysis in the presence of PO-DNA and PS-DNA confirmed by measuring the absorbance at 405 nm, and D) real-time absorbance data observed at 405 nm, where Blank represents a group that does not include nucleic acids.

FIG. 2 shows confirmation of optimal conditions for colorimetric analysis, showing A) an absorbance spectrum at 405 nm after 1 hour of reaction at room temperature for each hemin concentration (top-left), and a real-time absorbance curve for 1 hour (middle and right), and B) a result of colorimetric analysis for each H₂O₂ concentration, an absorbance spectrum at 405 nm after 1 hour of reaction at room temperature (top-left), and a real-time absorbance curve for 1 hour (middle and right).

FIG. 3 shows validation of effectiveness of PS bond, showing A) a result of analyzing absorbance according to the number of PS bonds introduced into a randomly selected identical sequence, B) a result of analyzing absorbance according to the presence or absence of single PS in consecutive nucleotides (A₂₀, T₂₀, C₂₀, and G₂₀) of the same length, and C) a result of analyzing absorbance according to the number of PS inserted into the sequence in consecutive nucleotides (A₂₀, T₂₀, and C₂₀) of the same length.

FIG. 4 shows results of absorbance analysis for experimental groups of FIG. 3, showing A) a result of real-time absorbance analysis for experimental groups of FIG. 3A, B) real-time absorbance curves for experimental groups of FIG. 3B, C) to E) real-time absorbance curves corresponding to FIG. 3C, and F) to I) real-time absorbance curves corresponding to FIG. 3.

FIG. 5 shows results of absorbance analysis of PW17 G-quadruplex according to a PS position, showing A) absorbance measurement results for 16 different sequences with different PS positions in the PW17 G-quadruplex sequence, B) a photograph of well plates after absorbance detection, and C) to D) real-time absorbance curves corresponding to FIG. 5A.

FIG. 6 shows comparison of differences in enzymatic activity in sequences with different lengths of consecutive bases consisting of the same base according to the presence or absence of PS, showing A), B), C), and D) analysis results for the types of bases A, T, C, and G, respectively.

FIG. 7 shows a comparative analysis of enzymatic activity according to proximal nucleobase compositions close to PS, showing A) an evaluation of the regulation of enzymatic activity through the interaction between the proximal nucleobases and PS, in which a library with total of 16 sequences consisting of 20 bases derived from TTTN*NTTTTTTTTN*NTTTTT and randomly arranged was created, and “N” represents bases A, T, G, and C, and the mixture was prepared as 80 pmole DNA. B) shows a library with total of 16 sequences that is ranked according to enzymatic activity results, and C) and D) show confirmation of the enzymatic activity for three different sequences generated based on a 0 PS sequence, and that 2 PS_H has higher enzymatic activity efficiency than 2 PS_L.

FIG. 8 shows A) and B) real-time absorbance curves and photographs of well plates corresponding to FIG. 7A, and C) real-time absorbance curves corresponding to FIG. 7C.

FIG. 9 shows results of sequence-specific colorimetric detection using a H-OSD probe, showing A) a schematic diagram of a difference in enzymatic activity according to DNA formation, B) a result of measuring absorbance according to the number of PS in dsDNA and ssDNA, C) an absorbance signal generation mechanism from a toehold-mediated strand displacement reaction, and D) a result of absorbance intensity at 405 nm from a plate reacted at room temperature for 1 hour using an enzymogenic signaling system.

FIG. 10 shows an optimal final MgSO₄ concentration for sequence-specific colorimetric diagnosis using an enzymogenic signaling system, analyzed using samples of FIG. 9D, showing A) absorbance spectra measured at 405 nm after 1 hour of reaction at room temperature, B) photographs of well plates after detection, and C) real-time absorbance curves for 1 hour.

FIG. 11 shows A) real-time absorbance curves corresponding to FIG. 9B, in which an ssDNA mixture was prepared and used with 20 pmoles of DNA (PO and PS), and a dsDNA mixture was prepared and used by annealing 20 pmole DNA (PO and PS) with 24 pmoles of complementary DNA (@PO-DNA), and PO-dsDNA and PS-dsDNA were hybridized before absorbance analysis. B) shows real-time absorbance curves corresponding to FIG. 9D.

FIG. 12 shows analysis of specificity of sequence-specific colorimetric diagnosis using an enzymogenic signaling system, in which a toehold-mediated strand displacement reaction was performed in the same method as in FIG. 9D, and 60 pmole of non-target DNA was used in the reaction. A) shows a result of absorbance measured at 405 nm for a plate reacted at room temperature for 1 hour, B) shows a result of real-time absorbance analysis measured at 405 nm, and C) shows a photograph of well plates after detection of an enzymogenic signaling system.

FIG. 13 shows analysis of an effect of the number of PS introduced into an H-OSD probe on enzymatic activity, in which a toehold-mediated strand displacement reaction was performed in the same method as in FIG. 9D, but this reaction was performed without including target DNA. A) shows a result of absorbance measured at 405 nm for a plate reacted at room temperature for 1 hour, B) shows a result of real-time absorbance analysis measured at 405 nm, and C) shows a photograph of well plates after detection of an enzymogenic signaling system.

FIG. 14 shows results of sequence-specific colorimetric detection using an OSD probe, in which A) is a schematic diagram showing a difference in enzymatic activity according to DNA formation, and B) shows results of absorbance measurement using an OSD probe containing different numbers of PS.

FIG. 15 shows an optimal final MgSO₄ concentration for sequence-specific colorimetric diagnosis using an enzymogenic signaling system for an OSD probe according to the present disclosure, showing A) absorbance spectra measured at 405 nm after 1 hour of reaction at room temperature, B) photographs of well plates after detection, and C) real-time absorbance curves for 1 hour.

FIG. 16 shows real-time absorbance curves for an OSD probe of the present disclosure according to the presence or absence of a target sequence and the number of PS.

FIG. 17 shows results of analyzing specificity of sequence-specific colorimetric diagnosis using an enzymogenic signaling system for an OSD probe according to the present disclosure by performing a toehold-mediated strand displacement reaction and using 60 pmole of non-target DNA in the reaction, showing A) a result of measuring the absorbance at 405 nm of a plate reacted at room temperature for 1 hour, B) a result of real-time absorbance analysis measured at 405 nm, and C) a photograph of well plates after detection of the enzymogenic signaling system.

### BEST MODE

The present disclosure relates to a sulfur-containing probe for nucleic acid-based colorimetric detection and a method for accelerating enzymatic activity through sulfur introduction into nucleic acids.

The present disclosure is characterized by providing a novel sulfur-containing probe for colorimetric detection that may improve problems of existing colorimetric analysis and accelerate enzymatic activity to enable rapid and accurate colorimetric detection.

Specifically, the sulfur-containing probe for colorimetric detection according to the present disclosure has the form of a DNA oligonucleotide in which nucleobases are continuously connected, and the continuous connection of nucleobases is characterized in that any one or more selected from adenine (A), guanine (G), cytosine (C), and thymine (T) are randomly connected, and a nucleotide phosphate backbone of the DNA oligonucleotide is modified with one or more phosphorothioates so as to include one or more sulfur atoms.

The sulfur-containing probe for colorimetric detection of the present disclosure is a new concept DNAzyme that is not subject to sequence limitations, unlike G-quadruplex used in existing colorimetric diagnosis, and is characterized by accelerating enzymatic activity mediated by a colorimetric reaction without requiring a sequence forming a specific structure such as G-quadruplex.

The sulfur-containing probe for colorimetric detection of the present disclosure may have the form of a DNA oligonucleotide in which 10 to 30 nucleobases are continuously connected, preferably the form of a DNA oligonucleotide in which 20 nucleobases are continuously connected.

In addition, the sulfur-containing probe for colorimetric detection of the present disclosure has a characteristic in that a sulfur element that does not exist in the original nucleic acid is introduced by modifying the phosphate backbone of a DNA oligonucleotide with phosphorothioates (PS: phosphorothioate modification), in which at least one of the nucleobases on either side of the PS may be adenine (A).

In one embodiment of the present disclosure, it was confirmed whether a sulfur atom affected a colorimetric reaction for the sulfur-containing probe for colorimetric detection of the present disclosure, and it was found that a signal response of absorbance was clearly observed in a probe into which a sulfur atom was introduced, whereas no signal response of absorbance was observed in a probe into which a sulfur atom was not introduced.

Further, in another embodiment of the present disclosure, it was analyzed whether the number of PS in which the phosphate backbone is modified with phosphorothioates affected the enzymatic activity of the colorimetric reaction. As a result, it was shown that the enzymatic activity increased as the number of PS increased, confirming that the number of PS affected the enzymatic activity.

In another embodiment, it was analyzed whether the length of the probe may affect the enzymatic activity, and as a result, no enzymatic activity was observed for monomers dNTPs and α-thio-dNTPs regardless of the type of nucleotide constituting the probe. Therefore, it was found that the length of the probe DNA may affect the enzymatic activity, which means that the degree of binding between DNA and hemin may vary depending on the length, and that only when DNA and hemin can bind, PS and nucleobases interact to show a colorimetric reaction.

Accordingly, the probe of the present disclosure for effective colorimetric detection may be a DNA oligonucleotide in which 10 to 30 nucleobases are continuously connected, and preferably a DNA oligonucleotide in which 20 nucleobases are continuously connected.

In addition, the sulfur-containing probe for colorimetric detection of the present disclosure is characterized in that the enzymatic activity varies depending on a type of nucleobase present in an adjacent position of PS. As a result of the experiment in Example of the present disclosure, when PS is positioned between adenine (A) and adenine (A) bases, the enzymatic activity efficiency was found to be the best compared to groups of combinations of other bases, and next, when any one of nucleobases adjacent to PS is adenine (A), the enzymatic activity was found to be excellent. On the other hand, when PS was positioned between pyrimidine bases such as T or C, the enzymatic activity ratio was relatively low.

Therefore, in order to derive an excellent colorimetric detection effect, when the phosphate backbone of the DNA oligonucleotide is modified with phosphorothioates, it is important that at least one of the nucleobases on either side of the modification is adenine (A). Preferably, the position of PS may be preferentially selected in the order of A*A, A*G, G*A, C*A, A*C, A*T, and T*A, and most preferably, PS may be positioned at A*A. The * mark indicates the position of PS.

In addition, the sulfur-containing probe for colorimetric detection of the present disclosure is characterized by having a difference in enzymatic activity according to a DNA form. That is, in the case of a probe in the form of single-stranded DNA, strong enzymatic activity is observed, but a probe in the form of double-stranded DNA is characterized in that enzymatic activity is reduced and thus no colorimetric reaction is observed.

Therefore, the sulfur-containing probe for colorimetric detection of the present disclosure has a form of single-stranded DNA.

Further, the present disclosure provides a composition for colorimetric detection including the sulfur-containing probe for colorimetric detection of the present disclosure.

The sulfur-containing probe for colorimetric detection provided in the present disclosure may act as a new DNAzyme, and the term “DNAzyme” typically means a nucleic acid molecule having enzymatic activity, and may, for example, have peroxidase activity. In addition, the DNAzyme may be a term including Deoxyribozymes, DNA enzymes, DNAzymes, catalytic DNA, DNA-based enzymes, etc., and may also be Ribonuclease and RNA ligase, and may also enable chemical reactions such as DNA phosphorylation, DNA adenylation, DNA deglycosylation, porphyrin metalation, thymine dimer photoreversion, DNA cleavage, etc.

Here, the DNAzyme may include a sulfur-containing probe sequence for colorimetric detection of the present disclosure.

The composition for colorimetric detection of the present disclosure may include a colorimetric reagent, and the colorimetric reagent may include, but is not limited to, at least one selected from the group consisting of 2,2′-azino-bis(3-ethylbenzothiazoline-6-sulfonic acid) (ABTS), o-phenylenediamine dihydrochloride (OPD), diaminobenzidine (DAB), 3-amino-9-ethylcarbazole (AEC), 3,3′,5,5′-tetramethylbenzidine (TMB), AmplexRed, and Homovanillic acid, and at least one peroxide. The peroxide may be, for example, hydrogen peroxide (H₂O₂).

In addition, the composition may include hemin as a DNAzyme cofactor.

In addition, the composition for colorimetric detection of the present disclosure may further include additional reagents and components used for colorimetric detection in the art.

In addition, the present disclosure may provide a kit for colorimetric detection including the composition for colorimetric detection of the present disclosure, and provide a colorimetric detection method using the sulfur-containing probe for colorimetric detection of the present disclosure.

Furthermore, the present disclosure may provide a method for preparing a sulfur-containing probe for colorimetric detection, having an effect of enhancing enzymatic activity mediated by a colorimetric reaction, and the method includes (1) selecting a target gene to be detected, (2) designing an oligonucleotide sequence complementary to a nucleic acid sequence of the target gene, and (3) modifying a nucleotide phosphate backbone in the complementary oligonucleotide with one or more phosphorothioates, in which the complementary oligonucleotide has a length of 10 to 30 mer, and the phosphorothioate modification is performed in a region in which at least one of nucleobases present on either side of the modification is adenine (A).

The sulfur-containing probe for colorimetric detection prepared by the method of the present disclosure accelerates enzymatic activity mediated by a colorimetric reaction to detect rapidly and accurately a target nucleic acid through colorimetric reaction analysis.

For example, in the case of using the probe of the present disclosure, when the nucleic acid to be detected is present in a sample, the absorbance value increases by the colorimetric reaction and a colorimetric signal is shown, whereas when the nucleic acid to be detected is not present in the sample, no colorimetric signal is shown.

In addition, the probe according to the present disclosure may be a sulfur-containing probe forming a hairpin structure, and may have a form designed as a loop structure composed of continuous T 10 mer, in which a single-stranded toehold sequence of 10 mer and a double-stranded stem structure of 20 bp are hybridized with each other at the 5′ or 3′ end of the sulfur-containing probe as complementary sequences to each other.

The probe of the present disclosure includes a sequence complementary to a target-binding site included in a target nucleic acid which is a single template strand, and the probe binds to the target nucleic acid from a toehold sequence, and branch migration of the probe structure occurs, resulting in a toehold-mediated strand displacement reaction that pushes out the originally hybridized complementary sequence.

In this case, a portion of the sequence including the toehold complementarily binds to the target nucleic acid and forms a new duplex structure through a hybridization process, and also, the conventional sequence that has been complementarily bound to the probe is freely released in a single-stranded form. The released single strand may include PS, and in the case of including PS, it was confirmed that the enzymatic activity was strongly shown in the same manner as the experimental results of the present disclosure above.

Therefore, the probe of the present disclosure may be applied and utilized not only as an H-OSD that forms a probe through intramolecular hybridization, but also as an OSD that forms a probe through intermolecular hybridization.

The OSD is composed of two complementary strands and may form a DNA duplex through intermolecular hybridization. The DNA duplex is divided into an output strand and a substrate strand released by single-strand displacement. The substrate strand, unlike the output strand, further includes a single-stranded toehold sequence.

When a complementary target nucleic acid is supplied, while the toehold sequence and the target nucleic acid partially bind to each other, the output strand is branch-migrated and then completely released. At this time, when the output strand that has been converted to a single strand state includes PS, the enzymatic activity may be strongly exhibited.

### [Modes]

Hereinafter, the present disclosure will be described in more detail with reference to Examples. However, these Examples are for more specifically illustrating the present disclosure, and the scope of the present disclosure is not limited to these Examples.

### <Preparation of Materials and Experimental Methods>

### Materials and Reagents

All oligonucleotides used in the experiment were synthesized from Bioneer (Daejeon, Republic of Korea) and Integrated DNA Technologies, Inc. (Coralville, IA, U.S.A.) and used. In addition, all DNA sequences used in the experiment are shown in Table 1 below. Citric acid, sodium citrate tribasic dihydrate, 2,2′-Azino-bis-(3-ethylbenzothiazoline-6-sulfonic acid)diammonium salt (ABTS), hemin, and dimethyl sulfoxide (DMSO) were purchased and used from Sigma, Inc. In addition, 27% (w/w) H₂O₂ were purchased and used from ThermoFisher Scientific, Inc. (Waltham, MA, USA). A 10× isothermal amplification buffer, and a deoxynucleotide (dNTP) solution set (100 mM) were purchased and used from New England Biolabs, Inc. (Beverly, MA, USA). A 96-well clear plate was prepared and used from Corning (Corning, NY, U.S.A.), α-thio-dATP, α-thio-dGTP, and α-thio-dCTP were purchased and used from TriLink Bio Technologies (San Diego, CA, USA), and dTTPαS (α-thio-dTTP) was prepared and used from Jena Bioscience GmbH (Jena, Germany). NaOH and HCl were purchased and used from iNtRON Biotechnology, Inc. (Seongnam, Korea), and a 2 mM Hemin stock solution was made using DMSO and stored at −20° C. In Table below, the * mark indicates phosphorothioate bond (PS) modification.

TABLE 1
A. Sulfur incorporation into random selected sequence according to the number of PS

| ID | Sequence (5′→3′) |
|---|---|
| PO-DNA 1 | ATA CAA AAC ATT CCC ACC AA |
| PS-DNA (1 PS)_1 | ATA CAA AAC A*TT CCC ACC AA |
| PS-DNA (2 PS)_1 | ATA CA*A AAC ATT CCC* ACC AA |
| PS-DNA (4 PS)_1 | ATA C* AA AA*C ATT* CCC A*CC AA |
| PS-DNA (8 PS)_1 | ATA* CA*A A*AC* AT*T C*CC* AC*C AA |

B. Effects of PS modification number, base type, and DNA length on enzymatic activity

| ID | Sequence (5′→3′) |
|---|---|
| A10 (PO) | AAA AAA AAA A |
| A₁₀ (1 PS) | AAA AA*A AAA A |
| A₂₀ (PO) | AAA AAA AAA AAA AAA AAA AA |
| A₂₀ (1 PS) | AAA AAA AAA A*AA AAA AAA AA |
| A₂₀ (2 PS) | AAA AAA* AAA AAA AA*A AAA AA |
| A₂₀ (4 PS) | AAA A*AA AA*A AAA* AAA A*AA AA |
| A₂₀ (8 PS) | AAA* AA*A A*AA* AA*A A*AA* AA*A AA |
| A₃₀ (PO) | AAA AAA AAA AAA AAA AAA AAA AAA AAA AAA |
| A₃₀ (1 PS) | AAA AAA AAA AAA AAA* AAA AAA AAA AAA AAA |
| T₁₀ (PO) | TTT TTT TTT T |
| T₁₀ (1 PS) | TTT TT*T TTT T |
| T₂₀ (PO) | TTT TTT TTT TTT TTT TTT TT |
| T₂₀ (1 PS) | TTT TTT TTT T*TT TTT TTT TT |
| T₂₀ (2 PS) | TTT TTT* TTT TTT TT*T TTT TT |
| T₂₀ (4 PS) | TTT T*TT TT*T TTT* TTT T*TT TT |
| T₂₀ (8 PS) | TTT* TT*T T*TT* TT*T T*TT* TT*T TT |
| T₃₀ (PO) | TTT TTT TTT TTT TTT TTT TTT TTT TTT TTT |
| T₃₀ (1 PS) | TTTTTT TTTTTT TTT* TTT TTT TTT TTT TTT |
| C₁₀ (PO) | CCC CCC CCC C |
| C₁₀ (1 PS) | CCC CC*C CCC C |
| C₂₀ (PO) | CCC CCC CCC CCC CCC CCC CC |
| C₂₀ (1 PS) | CCC CCC CCC C*CC CCC CCC CC |
| C₂₀ (2 PS) | CCC CCC* CCC CCC CC*CCCC CC |
| C₂₀ (4 PS) | CCC C*CC CC*C CCC* CCC C*CC CC |
| C₂₀ (8 PS) | CCC* CC*C C*CC* CC*C C*CC* CC*C CC |
| C₃₀ (PO) | CCC CCC CCC CCC CCC CCC CCC CCC CCC CCC |
| C₃₀ (1 PS) | CCC CCC CCC CCC CCC* CCC CCC CCC CCC CCC |
| Gio (PO) | GGG GGG GGG G |
| Gio (1 PS) | GGG GG*G GGG G |
| G20 (PO) | GGG GGG GGG GGG GGG GGG GG |
| G20 (1 PS) | GGG GGG GGG G*GG GGG GGG GG |

C. PW17 G-quadruplex

| ID | Sequence (5′→3′) |
|---|---|
| PW17 | GGG TAG GGC GGG TTG GG |
| PW17(PS)_1 | G*GG TAG GGC GGG TTG GG |
| PW17(PS)_2 | GG*G TAG GGC GGG TTG GG |
| PW17(PS)_3 | GGG* TAG GGC GGG TTG GG |
| PW17(PS)_4 | GGG T*AG GGC GGG TTG GG |
| PW17(PS)_5 | GGG TA*G GGC GGG TTG GG |
| PW17(PS)_6 | GGG TAG* GGC GGG TTG GG |
| PW17(PS)_7 | GGG TAG G*GC GGG TTG GG |
| PW17(PS)_8 | GGG TAG GG*C GGG TTG GG |
| PW17(PS)_9 | GGG TAG GGC* GGG TTG GG |
| PW17(PS)_10 | GGG TAG GGC G*GG TTG GG |
| PW17(PS)_11 | GGG TAG GGC GG*G TTG GG |
| PW17(PS)_12 | GGG TAG GGC GGG* TTG GG |
| PW17(PS)_13 | GGG TAG GGC GGG T*TG GG |
| PW17(PS)_14 | GGG TAG GGC GGG TT*G GG |
| PW17(PS)_15 | GGG TAG GGC GGG TTG* GG |
| PW17(PS)_16 | GGG TAG GGC GGG TTG G*G |

D. Enzymatic activity according to the influence of proximal base compositions close
to the PS

| ID | Sequence (5′→3′) |
|---|---|
| TC*CT | TTT C*CT TTT TTT TC*C TTT TT |
| TT*TT | TTT T*TT TTT TTT TT*T TTT TT |
| TA*AT | TTT A*AT TTT TTT TA*A TTT TT |
| TG*GT | TTT G*GT TTT TTT TG*G TTT TT |
| TA*GT | TTT A*GT TTT TTT TA*G TTT TT |
| TG*AT | TTT G*AT TTT TTT TG*A TTT TT |
| TA*TT | TTT A*TT TTT TTT TA*T TTT TT |
| TT*AT | TTT T*AT TTT TTT TT*A TTT TT |
| TA*CT | TTT A*CT TTT TTT TA*C TTT TT |
| TC*AT | TTT C*AT TTT TTT TC*A TTT TT |
| TG*TT | TTT G*TT TTT TTT TG*T TTT TT |
| TT*GT | TTT T*GT TTT TTT TT*G TTT TT |
| TG*CT | TTT G*CT TTT TTT TG*C TTT TT |
| TC*GT | TTT C*GT TTT TTT TC*G TTT TT |
| TT*CT | TTT T*CT TTT TTT TT*C TTT TT |
| TC*TC | TTT C*TT TTT TTT TC*T TTT TT |

E. Enzyme activity according to the controlled PS position in the same sequence

| ID | Sequence (5′→3′) |
|---|---|
| 0 PS | GGA AGA CCT TAA ATT CCC TCG |
| 2 PS_L | GGA AGA CC*T TAA ATT* CCC TCG |
| 2 PS_M | GGA* AGA CCT TAA ATT* CCC TCG |
| 2 PS_H | GGA* AGA CCT TAA* ATT CCC TCG |

F. Enzyme activity according to DNA formation

| ID | Sequence (5′→3′) |
|---|---|
| PO-DNA 2 | GGA AGA CCT TAA ATT CCC TCG |
| PS-DNA (4 PS)_2 | GG*A AG*A CCT T*AA* ATT CCC TCG |
| PS-DNA (6 PS)_2 | GG*A A*G*A CCT T*A*A* ATT CCC TCG |
| PS-DNA (8 PS)_2 | GG*A* A*G*A C*CT T*A*A* ATT CCC TCG |
| ©PO-DNA | CGA GGG AAT TTA AGG TCT TCC |

G. Sequence-specific colorimetric diagnosis using H-OSD probe

| ID | Sequence (5′→3′) |
|---|---|
| Target DNA | TTT TTG GAA GAC CTT AAA TTC CCT CGA GGA CAA G |
| | GC GTT CCA ATT ATT TTT |
| H-OSD (PO) | GCC TTG TCC TCG AGG GAA TTT AAG GTC TTC CTT TT |
| | T TTT TTG GAA GAC CTT AAA TTC CCT CG |
| H-OSD (4 PS) | GCC TTG TCC TCG AGG GAA TTT AAG GTC TTC CTT TT |
| | T TTT TTG G*AA G*AC CTT* AA*A TTC CCT CG |
| H-OSD (6 PS) | GCC TTG TCC TCG AGG GAA TTT AAG GTC TTC CTT TT |
| | T TTT TTG G*AA* G*AC CTT* A*A*A TTC CCT CG |
| H-OSD (8 PS) | GCC TTG TCC TCG AGG GAA TTT AAG GTC TTC CTT TT |
| | T TTT TTG G*A*A* G*AC* CTT* A*A*A TTC CCT CG |

H. Sequence-specific colorimetric diagnosis using OSD probe

| ID | Sequence (5′→3′) |
|---|---|
| Target DNA | TTT TTA TCA CGT AGT CGC AAC AGT TCA AGA AAT TCT TTT T |
| Toehold region | GAA TTT CTT GAA CTG TTG CGA CTA CGT GAT |
| Release region(PO) | CGC AAC AGT TCA AGA AAT TC |
| Release region(2PS) | CGC A*AC AGT TCA AGA* AAT TC |
| Non-target DNA | TTT TTC AAA CTG TCA CTA AGA AAT CTG CTG CTG AGT TTTT |

### Mononucleotide and DNA Preparation

All DNA mixtures were prepared in a 1× isothermal amplification buffer (20 mM Tris-HCl, 10 mM (NH₄)₂SO₄, 50 mM KCl, 2 mM MgSO₄, 0.1% Tween® 20, pH 8.8). An ssDNA mixture was prepared at 40 pmole of DNA, and a mononucleotide mixture was prepared at 40 pmole of dNTP or α-thio-dNTP. All nucleic acids were used at a concentration of 40 pmole unless otherwise noted. The DNA mixture was reacted at 95° C. for 5 minutes and then slowly cooled to 25° C. at a rate of 0.1° C./s, and thereafter, the DNA mixture was kept on 25° C. at least 5 minutes before use.

### PW17 G-Quadruplex Analysis

A G-quadruplex analyte was prepared in a 1× isothermal amplification buffer (20 mM Tris-HCl, 10 mM (NH₄)₂SO₄, 50 mM KCl, 2 mM MgSO₄, 0.1% Tween® 20, pH 8.8). The PW17 mixture was prepared by reacting with 40 pmole DNA (PO or 1 PS), and the analyte was reacted at 95° C. for 5 minutes and then slowly cooled to 25° C. at a rate of 0.1° C./s, and reacted at room temperature for 5 minutes.

### Absorbance Measurement and Photograph Acquisition

The total volume of an analytical sample was set to 122.5 μL. 1.5 μL of 75 μM hemin, 100 μL of a 100 mM citrate buffer (pH 4.0 at 25° C.) containing 0.025% (w/v) 2,2′-Azino-bis-(3-ethylbenzothiazoline-6-sulfonic acid) diammonium salt (ABTS), and 1 μL of 3% H₂O₂ were added to 20 μL of a DNA mixture. Then, 120 μL of a final sample solution was transferred into a 96-well clear plate, and reacted at 24 to 25° C. for 1 hour. Thereafter, the absorption intensity at 405 nm of the reaction mixture was recorded every 1 minute for a total of 60 times using a Tecan Spark® M10 multimode microplate reader (Männedorf, Switzerland). In addition, images were acquired by loading the analytical sample on a 96-well clear plate (Corning cat. No. 9017) and then photographing using EOS-7D (Canon, Japan).

### Enzymogenic Probe Preparation

A probe solution was prepared by annealing 25 UM of a hairpin probe strand in a 1× Isothermal amplification buffer (20 mM Tris-HCl, 10 mM (NH₄)₂SO₄, 50 mM KCl, 2 mM MgSO₄, 0.1% Tween® 20, pH 8.8), and 6 mM MgSO₄ (final 8 mM). Thereafter, the probe solution was reacted at 95° C. for 5 minutes, slowly cooled to 25° C. at a rate of 0.1° C./s, and then reacted at room temperature for 1 hour, and the probe was stored at 4° C. before use for the experiment.

### Enzymogenic Probe Preparation-OSD

A probe solution was prepared by annealing a 25 μM release strand and a 30 μM toehold strand in a 1× isothermal amplification buffer (20 mM Tris-HCl, 10 mM (NH₄)₂SO₄, 50 mM KCl, 2 mM MgSO₄, 0.1% Tween® 20, pH 8.8), and 6 mM MgSO₄ (final 8 mM). Thereafter, the probe solution was reacted at 95° C. for 5 minutes, slowly cooled to 25° C. at a rate of 0.1° C./s, and then reacted at room temperature for 1 hour, and the probe was stored at 4° C. before use for the experiment.

### Sequence-Specific Colorimetric Analysis Using H-OSD and OSD Probes

A 1× isothermal amplification buffer (20 mM Tris-HCl, 10 mM (NH₄)₂SO₄, 50 mM KCl, 2 mM MgSO₄, 0.1% Tween® 20, pH 8.8), 6 mM MgSO₄ (final 8 mM), 60 pmole of a target DNA, and 1.6 μL of a probe solution were added to initiate toehold-mediated strand displacement reactions, and the reactions were performed at 25° C. for 1 hour. Thereafter, 1.5 μL of 75 μM hemin, 100 μL of a 100 mM citrate buffer (pH 4.0) containing 0.025% (w/v) 2,2′-Azino-bis-(3-ethylbenzothiazoline-6-sulfonic acid) diammonium salt (ABTS), and 1 μL of 3% H₂O₂ were added and mixed to 20 μL of a reaction product. 120 μL of the final sample solution was transferred into a 96-well clear plate, and maintained at 24° C. for 1 hour. Thereafter, the absorption intensity at 405 nm of the reaction mixture was recorded every 1 minute for a total of 60 times using a Tecan Spark® M10 multimode microplate reader (Männedorf, Switzerland). The analytical samples loaded on the 96-well clear plate (Corning cat. No. 9017) were photographed using EOS-7D (Canon, Japan).

### Example 1

Introduction of Sulfur Atoms into Single-Stranded DNA (ssDNA)

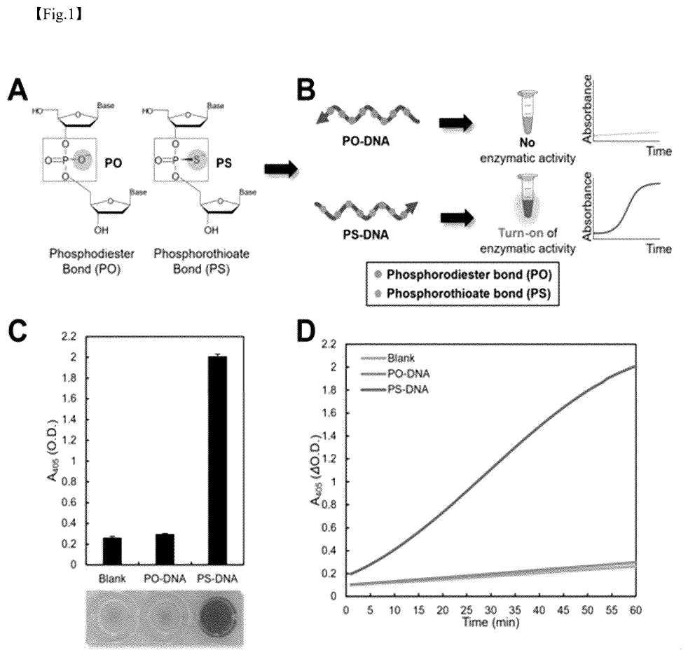

When sulfur atoms which were not intrinsically included in nucleic acids were introduced into the nucleic acids through phosphorothioate bond (PS) modification, an effect of the sulfur atoms on a colorimetric reaction was analyzed (FIG. 1A). At this time, the sulfur atoms may be included in various forms of nucleic acids.

As a result, when the PS was introduced into ssDNA, enzymatic activity was confirmed and the enzymatic activity was activated, so that a high absorbance (O.D.) signal response was observed. Conversely, there was almost no absorbance signal response in phosphodiester-bond ssDNA (FIG. 1B).

Thus, the present inventors confirmed that when a modification was induced by introducing sulfur atoms into a single-stranded DNA backbone, enzymatic activity was able to be simply and conveniently activated even in the absence of a sequence forming a specific structure such as G-quadruplex.

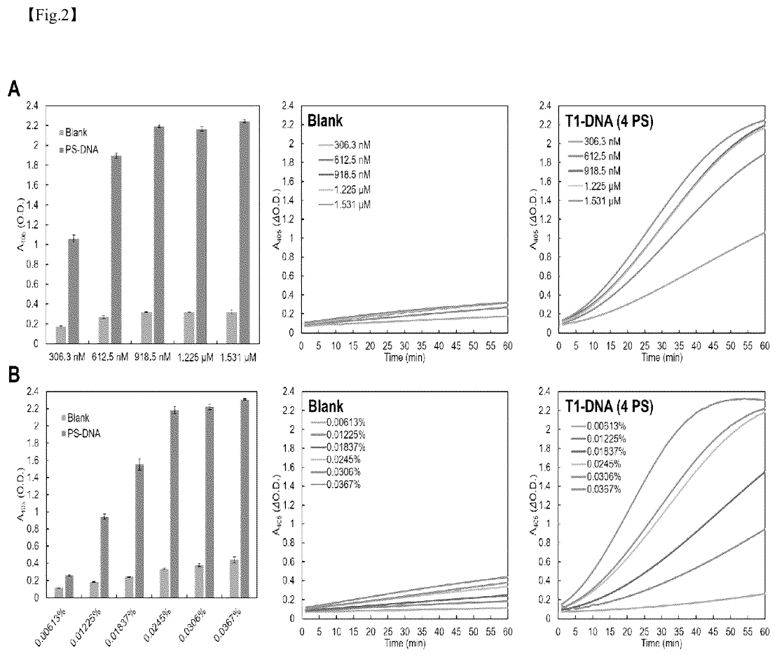

In addition, a phosphorothioate bond (PS), as an artificial linkage modification introducing sulfur into nucleic acids, was introduced into a specific sequence to observe an effect of enzymatic activity. At this time, in an N gene of a SARS-COV-2 virus, a 20 mer oligonucleotide (PO-DNA: 5′ATACAAAACATTCCCACCAA-3′) was selected as the specific sequence. In addition, DNA (PS-DNA: 5′-ATAC*AAAA*CATT*CCCA*CCAA-3′) inducing PS bond modification was prepared for the same specific sequence, and at this time, the PS was designed to be evenly distributed into the sequence, and the asterisk (*) indicates a sulfur introduction site. In addition, to maximize a color change in the presence of PS-DNA, the concentrations of hemin and H₂O₂ were optimized (FIG. 2), and the concentrations of the citrate buffer and ABTS were used as established. Each of PO-DNA and PS-DNA was mixed with a reaction buffer containing hemin, H₂O₂, and ABTS, and then the color change was monitored in real time by measuring the absorbance intensity.

As a result, after a few minutes, different absorbance signal response results started to be shown between PO-DNA and PS-DNA, and the absorbance significantly increased in the presence of PS-DNA up to around 2.0, whereas the absorbance was similar to that of a negative control group (Blank) in a PO-DNA treated group (FIGS. 1C and 1D).

Through these colorimetric analysis results, the present inventors were able to determine that when DNA in which sulfur atoms have been modified with PS was used as a probe for colorimetric detection, enzymatic activity was accelerated to derive more rapid and accurate colorimetric diagnostic results.

### Example 2

### Analysis of Effects of PS Modification on Enzymatic Activity

To find out how the enzymatic activity changes with the number of PS, the present inventors conducted an experiment by introducing various number of PS (0, 1, 2, 4, 8) into nucleic acids.

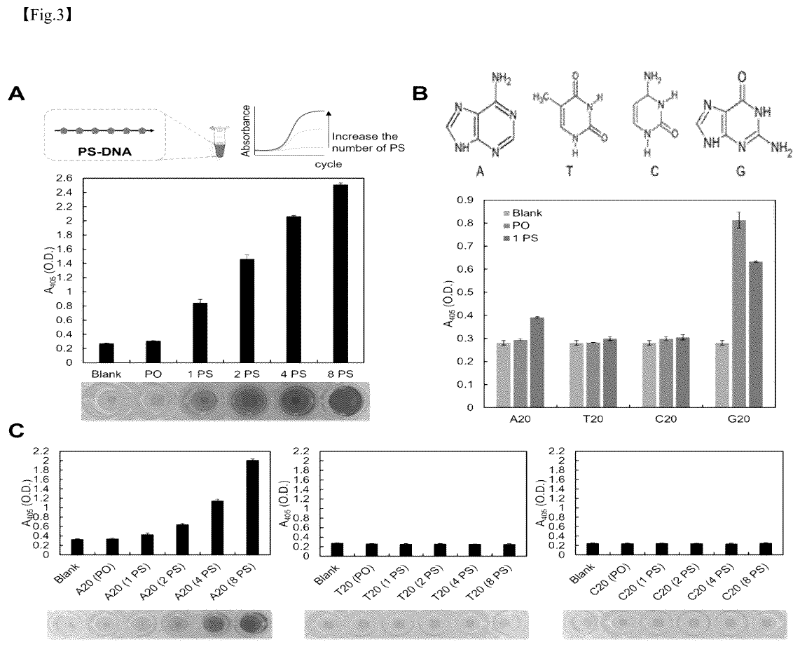

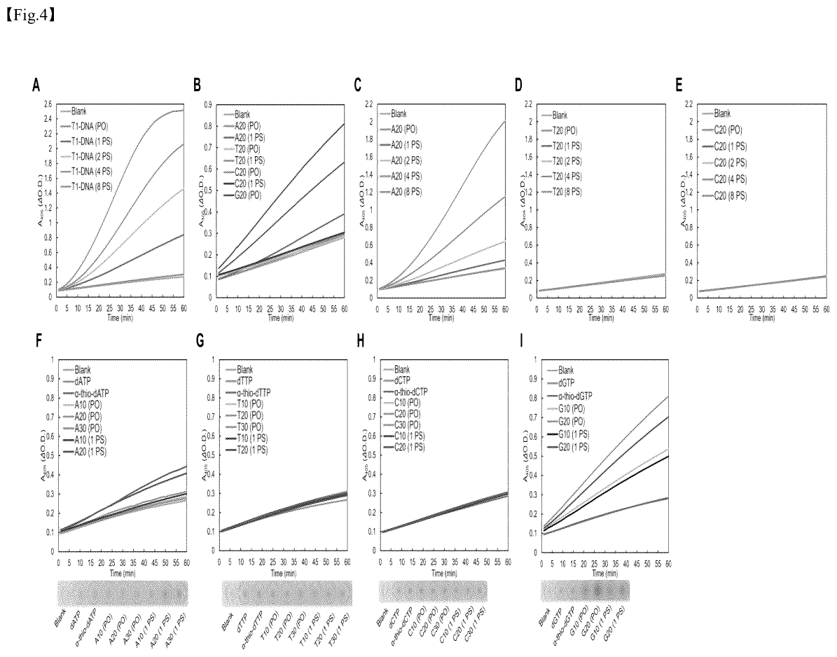

As a result, it was confirmed that as the number of PS increased, the enzymatic activity increased (FIGS. 3A and 4A). This means that as the number of PS included in the nucleic acids increased, the enzymatic activity increased. This result found that PS positively contributed to the occurrence of enzymatic activity and that the number of PS played an important role in enzymatic activity.

Next, the present inventors analyzed a correlation between PS and nucleobases. To this end, sequences with 20 consecutive nucleotides for each of A, C, G, and T nucleotides were prepared, and experiments were conducted with and without introduced PS. For reference, A₂₀ refers to a nucleotide consisting of 20 consecutive adenine (A) bases.

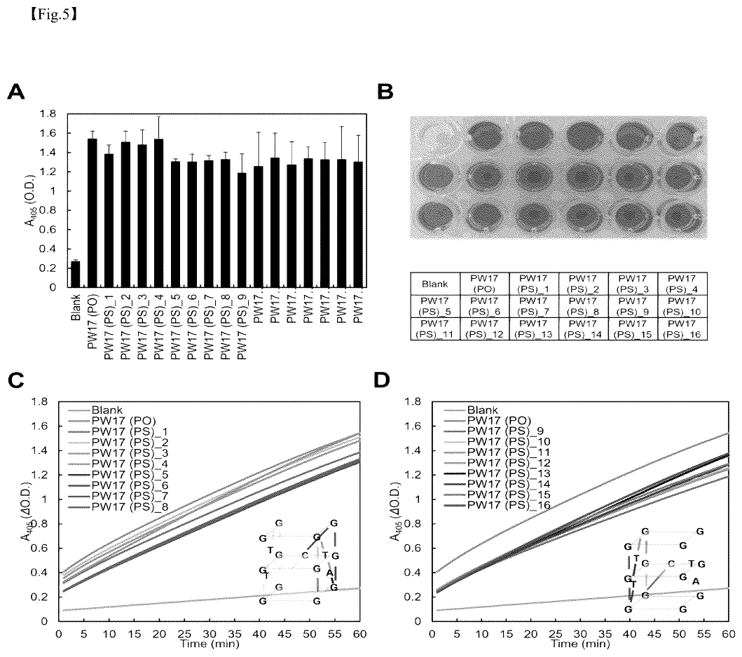

As a result, it was shown that the absorption values increased in A₂₀ and G₂₀ nucleotides when PS was present, whereas the absorption values did not change significantly in C₂₀ and T₂₀. In addition, unlike other types of nucleotides, in the case of G₂₀, it was shown that the enzymatic activity increased with and without PS compared to a blank (FIGS. 3B and 4B), which was because a G-quadruplex structure may be partially formed even without PS, leading to relatively increased enzyme activity. In addition, when PS was introduced, the trend in which the enzymatic activity decreased compared to PO showed that the addition of PS increased the structural instability during the formation of intermolecular G-quadruplexes, thereby decreasing the enzymatic activity. In addition, the effect of PS on standard and well-known G-quadruplex structure was further confirmed, but no significant effect was observed (FIG. 5). On the other hand, although the effect of A₂₀ was not dramatic, a slight increase in absorbance value was observed. Unlike G₂₀, A₂₀ did not form a specific structure, so that the enzymatic activity was shown with ssDNA alone. Through this, it was determined that purine bases were more efficiently involved in enzymatic activity than a pyrimidine base.

In addition, the present inventors confirmed whether the enzymatic activity increased when the number of PS increased, and at this time, the G₂₀ sequence was not used in this analysis due to self-assembly characteristics (e.g., intermolecular G-quadruplex). In the case of C₂₀ and T₂₀, the enzymatic activity did not increase despite increasing the number of PS up to 8. On the other hand, in the case of A₂₀, it was confirmed that the enzymatic activity increased as the number of PS increased (FIGS. 3C and FIGS. 4C to 4E). These results showed that PS had a more effective effect on nucleobases, particularly adenine, and that the amount of PS also played an important role.

In addition, the present inventors used monomeric nucleotides and single-stranded oligonucleotides, to investigate whether the length of the nucleotide affected the enzymatic activity for each of four DNA nucleotides A, C, G, and T. dNTP and α-thio-dNTP were used as monomeric nucleotides. The general form of dNTP is the original form containing an oxygen atom in α-phosphate, whereas α-thio-dNTP refers to the form in which a sulfur atom is introduced instead of a non-crosslinking oxygen atom in α-phosphate. A single oligonucleotide used a repeat sequence formed by consecutively repeating each base to be 10 mer, 20 mer, and 30 mer for A/C/T, and a repeat sequence of 10 mer and 20 mer for G. In addition, a single oligonucleotide containing PS was prepared and used by introducing PS in the middle of each DNA sequence.

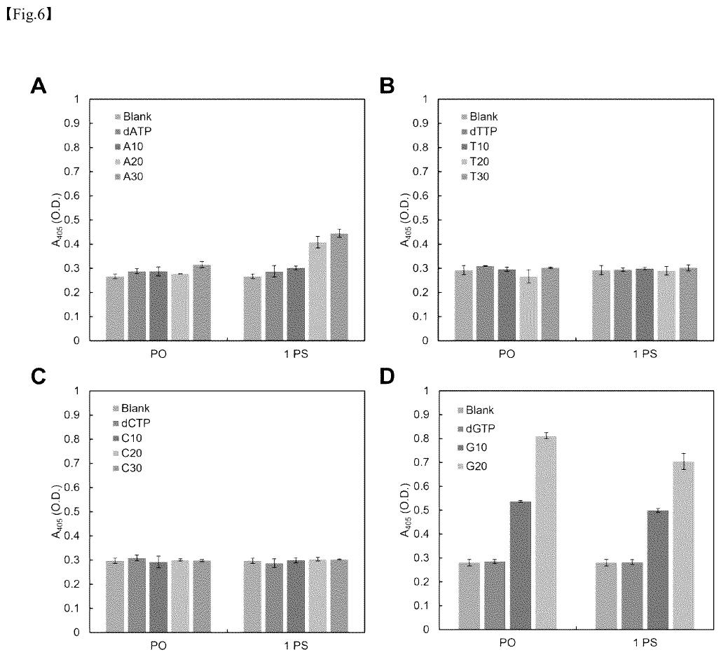

As a result, in the case of monomers dNTP and α-thio-dNTP, enzymatic activity was not observed regardless of the type of nucleotide. In addition, it was shown that the enzymatic activity increased as the length of adenine (A) increased, which was verified that a DNA length had a positive effect on the binding of DNA and hemin. From these results, it was determined that the binding degree of DNA and hemin varied with a length, and PS and nucleobases interact with each other, only when DNA and hemin can bind. That is, in the case of a monomer into which a sulfur atom has been introduced, even if the sulfur atom has been introduced, the monomer may not bind to DNA and hemin to exhibit enzymatic activity. In addition, it may be seen that ssDNA may bind to a porphyrin ring of hemin through a π-π stacking interaction, and that as the length is increased, DNA and hemin may more stably bind, which may induce high enzymatic activity (FIG. 6 and FIGS. 4F to 4I).

### Example 3

### Analysis of Effect of Proximal Nucleotide Combination in Adjacent Position of PS on Enzymatic Activity

Through the experiments of the Examples above, it was confirmed that DNA and hemin form a π-π stacking interaction and that enzymatic activity is accelerated by the influence of PS. In addition, it was confirmed that enzymatic activity varies depending on the length of DNA and the types of nucleobases composing the DNA. Therefore, the present inventors selected thymine (T) as a nucleotide backbone to evaluate enzymatic activity for various combinations of nucleobases. An analysis based on a continuous T sequence with low electron density could clearly confirm enzymatic activity for various combinations of nucleobases adjacent to PS. T serves primarily as a spacer in the G-quadruplex structure and does not significantly contribute to actual efficiency enhancement. However, in sequences with a high proportion of T, the overall enzymatic activity may be weak. Therefore, a combination of nucleobases at positions 4 and 5 and nucleobases at positions 14 and 15 was repeated and included in a T-based nucleotide sequence, and 16 different T-based sequences, each 20-nt in length, were prepared and used by including one PS between the nucleobases at positions 4 and 5 and between the nucleobases at positions 14 and 15, respectively (see Table 2 below).

TABLE 2
Ranking list of base combination

| | | |
|---|---|---|
| TA*AT | TTTA*ATTTTTTTTA*ATTTTT | 1 |
| TA*GT | TTTA*GTTTTTTTTA*GTTTTT | 2 |
| TG*AT | TTTG*ATTTTTTTTG*ATTTTT | 3 |
| TC*AT | TTTC*GTTTTTTTTC*GTTTTT | 4 |
| TA*CT | TTTA*GTTTTTTTTA*GTTTTT | 5 |
| TA*TT | TTTA*TTTTTTTTTA*TTTTTT | 6 |
| TC*GT | TTTC*GTTTTTTTTC*GTTTTT | 7 |
| TT*AT | TTTT*ATTTTTTTTT*ATTTTT | 8 |
| TG*GT | TTTG*GTTTTTTTTG*GTTTTT | 9 |
| TC*CT | TTTC*CTTTTTTTTC*CTTTTT | 10 |
| TT*GT | TTTT*GTTTTTTTTT*GTTTTT | 11 |
| TG*TT | TTTG*TTTTTTTTTG*TTTTTT | 12 |
| TT*CT | TTTT*CTTTTTTTTT*CTTTTT | 13 |
| TG*CT | TTTG*CTTTTTTTTG*CTTTTT | 14 |
| TT*TT | TTTT*TTTTTTTTTT*TTTTTT | 15 |
| TC*TT | TTTC*TTTTTTTTTC*TTTTTT | 16 |

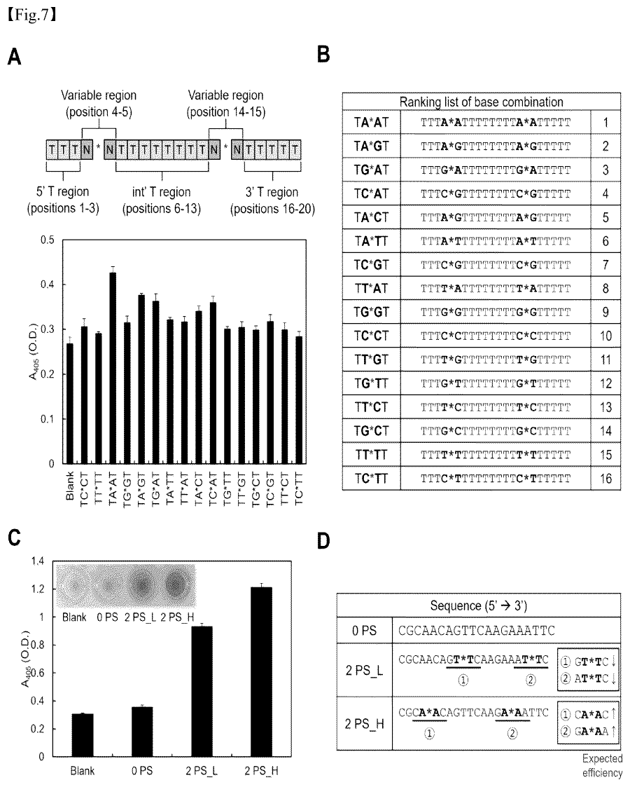

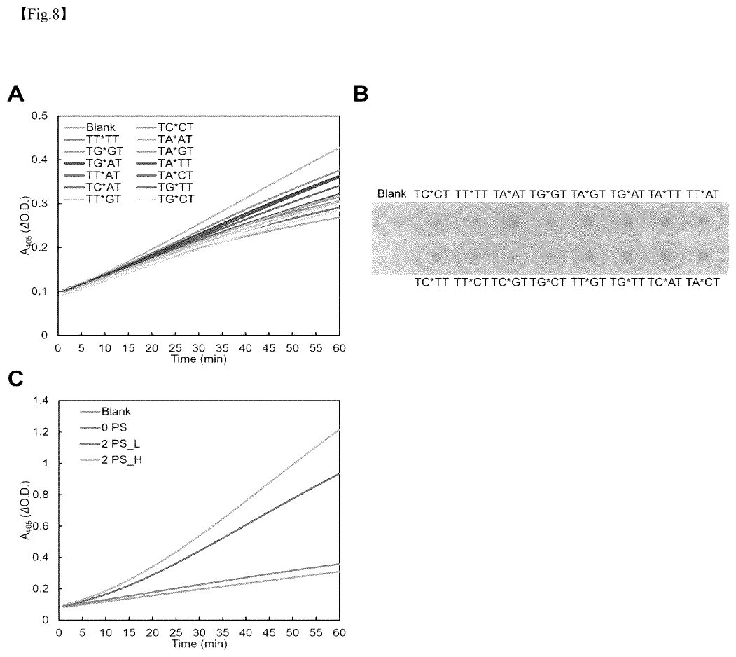

As a result, it was shown that the enzymatic activity varied depending on a type of base combination adjacent to the PS and the order of bases on either side of the PS, and the sequence ranking according to enzymatic activity efficiency was shown in Table 2 above and FIG. 7A. Specifically, among 16 sequences, AA was found to exhibit the highest efficiency, and an A base, which constituted AA, was identified as a key base that enhances enzymatic activity of G-quadruplex. Through this, the present inventors were able to determine that among the nucleobases. A may be used as a priority base at a position where PS was inserted to exhibit enzymatic activity. In addition, in the ranking list, AN or NA was ranked high, which indicated that heterocombinations containing A may also be used next to AA. In contrast, GG showed a relatively low rank despite being a purine base like A, and GX (X=T, C, G) or X*G showed an even lower rank, excluding combinations such as AG and GA. This was shown because G had a lower reduction potential than A. It was confirmed that purine bases have a greater number of electrons that may participate in enzymatic activity, and combinations of purine and pyrimidine bases may also exhibit relatively good efficiency. Sequences composed solely of pyrimidines were found at the bottom of the ranking list and were shown to have lower enzymatic activity, because pyrimidine bases such as T or C generally have a lower electron density than purines (FIGS. 7A and 8A to 8B).

Furthermore, the present inventors selected and used a random DNA sequence having a 50% GC content that did not form a secondary structure, instead of the sequence used in FIG. 1. In the Examples above, even when only one PS was introduced, the random sequence showed significant enzymatic activity (FIG. 3A). In this experiment, to distinguish sufficient efficiency, a sequence introduced with two PS was used. According to the ranking of base combinations of Table 2 above, combinations that exhibited the highest efficiency were selected to be introduced with PS. Furthermore, positions of combinations that exhibited low efficiency within the same sequence, were selected to be introduced with PS and then two sequences designated as low efficiency (L) and high efficiency (H) of FIG. 3 were used in the experiment.

TABLE 3
Sequence (5′ → 3′)

| | |
|---|---|
| 0 PS | GGAAGACCTTAAATTCCCTCG |
| 2 PS_L | 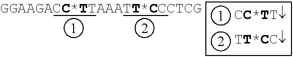 |
| 2 PS_H | 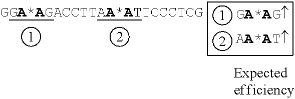 |

The analysis results showed that the enzymatic activity was excellent when the bases adjacent to PS were AA combinations, similar to the results in FIGS. 7A and 7B (FIGS. 7C to 7D and FIG. 8C). Through this, it was confirmed that for effective colorimetric detection, it was advantageous to introduce the PS in positions where at least one of the bases adjacent to PS is adenine (A).

### Example 4

### Sequence-Specific Colorimetric Analysis Using Enzymogenic Signaling System

Through the results of Examples above, it was confirmed that the introduction of PS into ssDNA strongly enhanced enzymatic activity. This process is performed through the following steps. First, hemin binds to ssDNA of an appropriate length. Second, PS increases the electron density near the hemin-bound base. Third, the heightened electron density in the bases accelerates enzymatic activity by interacting with hemin. Here, the bases play a direct role in accelerating the enzymatic activity of hemin.

Accordingly, the present inventors used dsDNA formed by binding a complementary sequence to ssDNA to compare the enzymatic activity with respect to a difference in nucleic acid form between ssDNA and dsDNA.

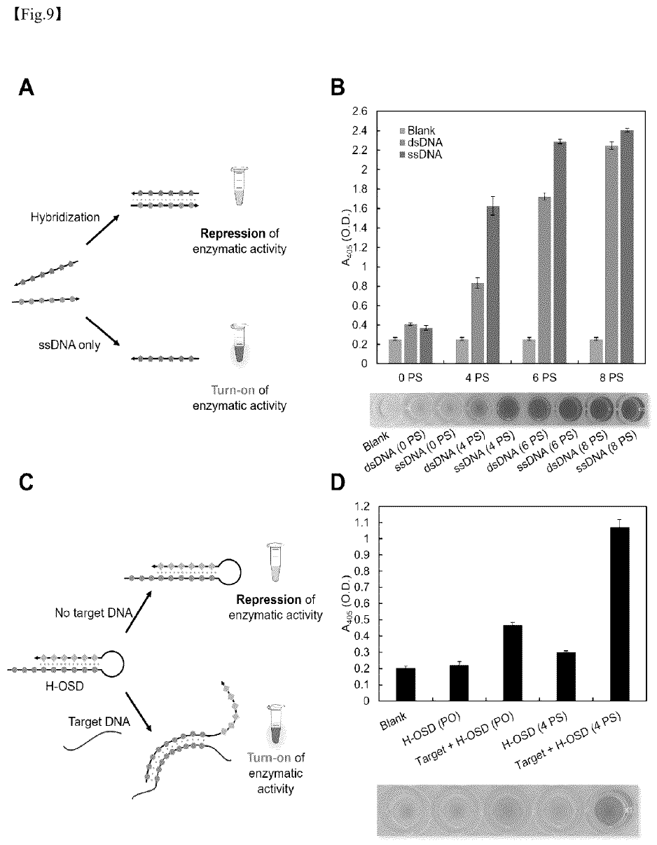

As a result, it was shown that the enzymatic activity was strong in the PS-ssDNA form, but the enzymatic activity decreased when complementary ssDNA was bound to form dsDNA, and also that the enzymatic activity was enhanced again when a specific target DNA or RNA interacted within the dsDNA to release a PS-ssDNA portion (FIG. 9). By using the mechanism, it is possible to construct an enzymogenic system that may be activated by target DNA or RNA.

In addition, to verify this content, the present inventors prepared complementary sequences to the sequences used in Table 2 above, and evaluated enzymatic activity after undergoing appropriate DNA hybridization conditions.

As a result, it was observed through absorbance analysis that enzymatic activity decreased in dsDNA containing PS. Meanwhile, it was found that as the number of PS increased, the repression efficiency of enzymatic activity in dsDNA decreased (FIGS. 9A and 9B). The reason was that instability in overall DNA hybridization was increased due to DNA end breathing in intermolecular DNA hybridization within dsDNA and the influence of PS, potentially leading to the exposure of bases from both ssDNA strands. This increased instability might have elevated the possibility of unwanted enzymatic activity, because hemin, H₂O₂, and ABTS could interact with the exposed bases.

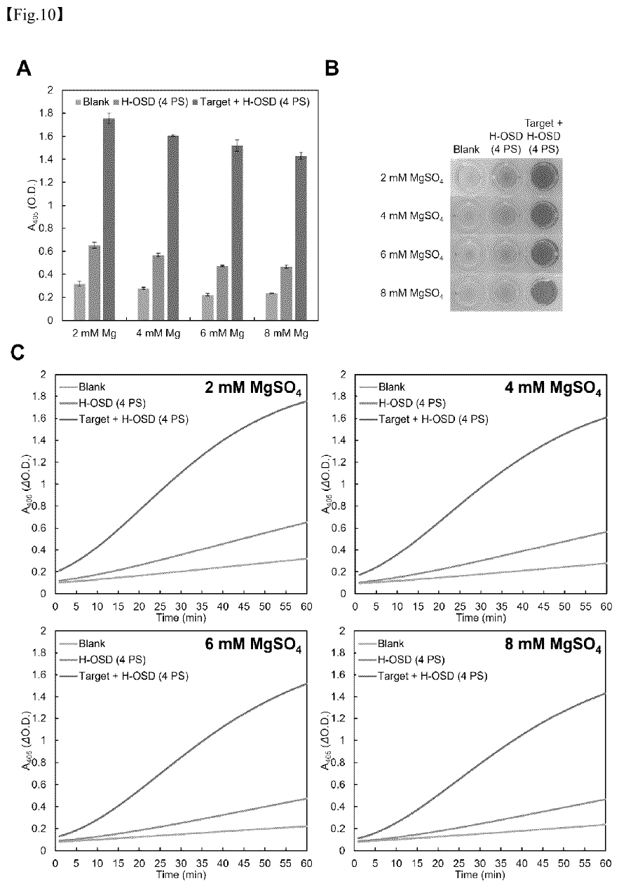

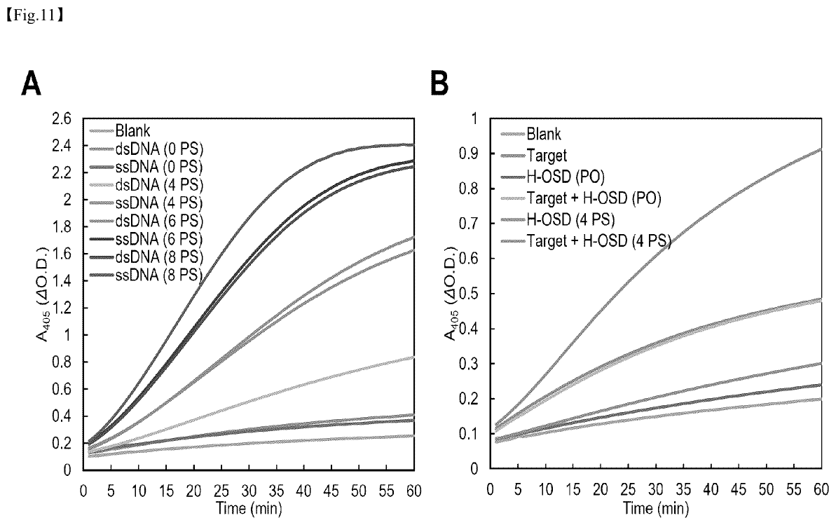

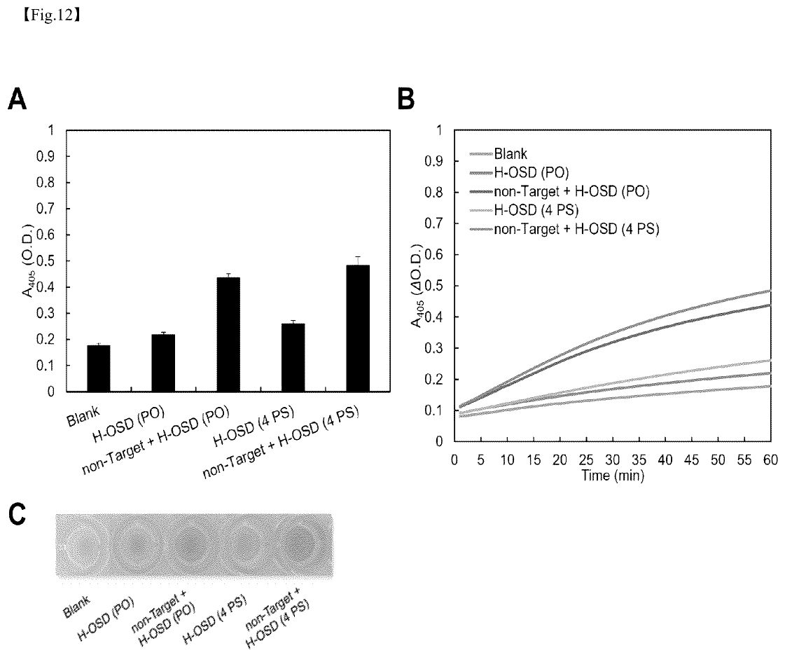

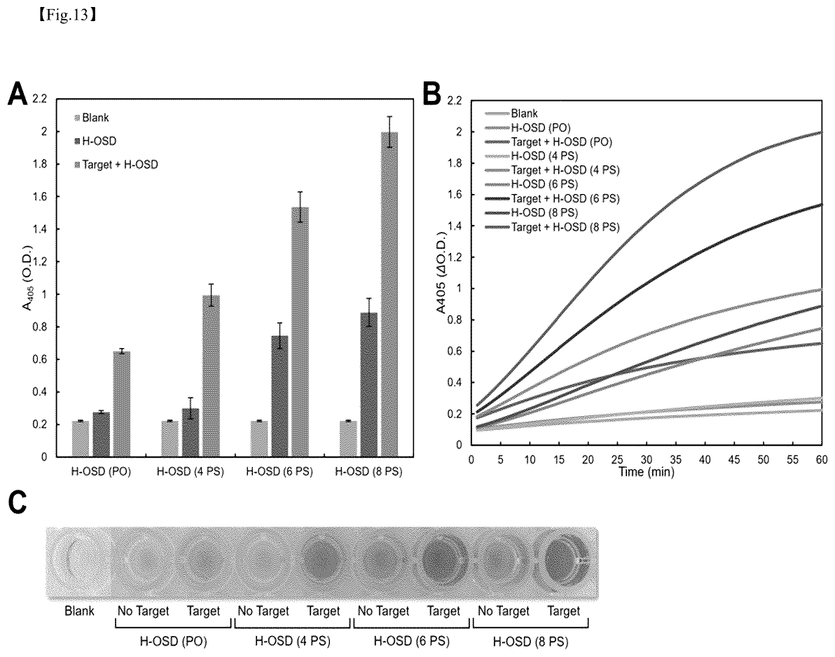

Furthermore, to mitigate the potential for signal leakage due to intermolecular DNA hybridization, the probe for the enzymogenic signaling system was designed in a hairpin format, termed H-OSD (intramolecular DNA hybridization). In this configuration, an appropriate length hairpin stem was selected and used to ensure stability of a duplex region and sufficient enzymatic activity, even when multiple PS molecules were introduced. The H-OSD probe consisted of a 21 bp hairpin stem with a 10-nt toehold at the 5′-end, which was complementary to a target sequence up to a loop sequence. A toehold region hybridized with the target sequence to open the strand containing PS and activate enzymatic activity. Conversely, in the absence of the target sequence, enzymatic activity remained inactive. In this state, the hairpin structure was maintained, and the loop sequence consisted only of T to avoid background increases even when exposed (FIGS. 9C and 9D). Additional conditions were optimized to achieve an optimal absorbance gradient and prevent the risk of signal leakage induced by DNA instability for target presence or absence (FIG. 10). Further, even in the presence of non-target sequences, the hairpin remained closed, and only introduced non-target signals were detected (FIGS. 11 and 12). In addition, a background control capability was evaluated by increasing the number of PS molecules in the H-OSD probe up to 8, and it was observed that intramolecular DNA hybridization was more stable and less affected by the increased dsDNA instability caused by the addition of PS (FIG. 13).

### Example 5

### Sequence-Specific Colorimetric Diagnosis Using Enzymogenic Signaling System

Furthermore, the present inventors prepared a probe composed in a different manner from Example 4. In Example 4, a hairpin-shaped H-OSD formed through intramolecular hybridization was utilized for sequence-specific colorimetric detection. In contrast, in Example 5, when a sulfur atom was introduced into a probe formed through intermolecular DNA hybridization using PS modification, the effect of enhancing activation of enzymatic activity due to the release of a single strand including PS was analyzed.

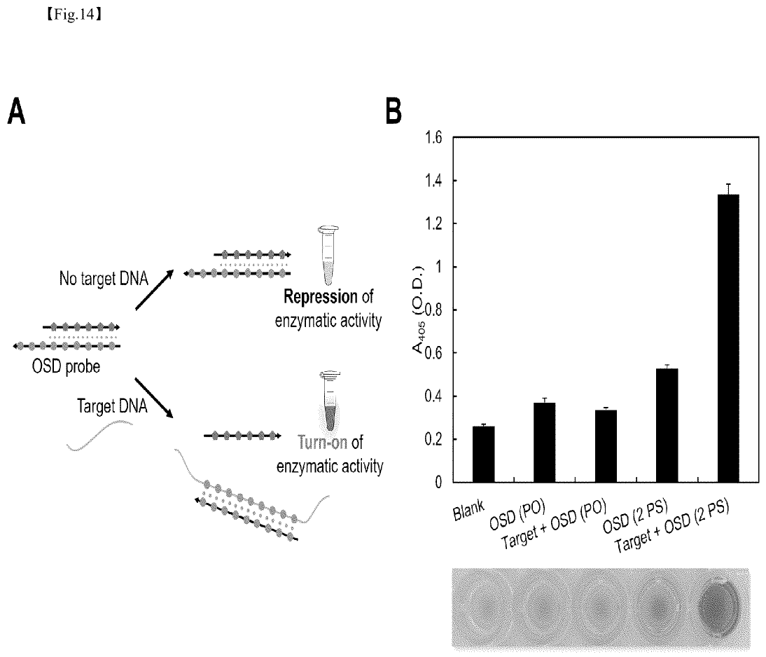

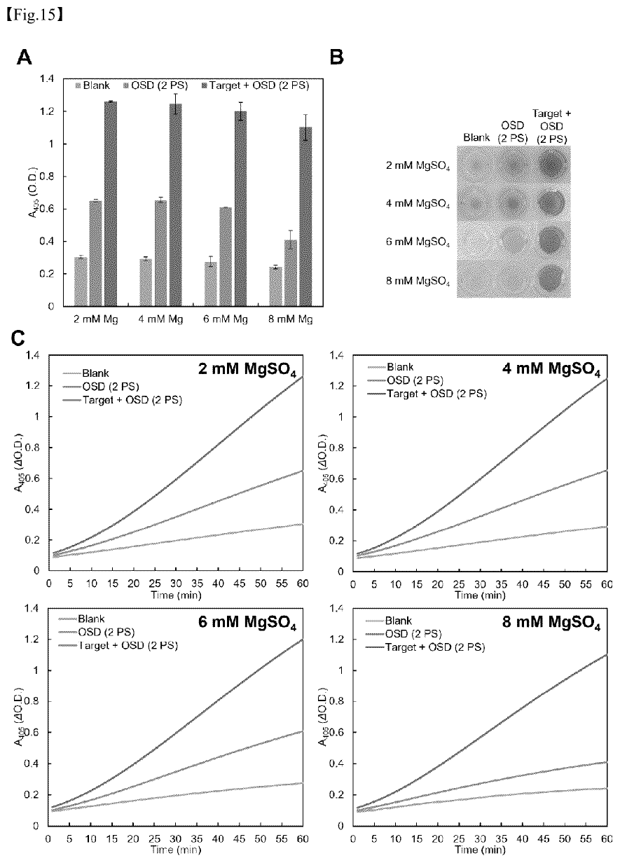

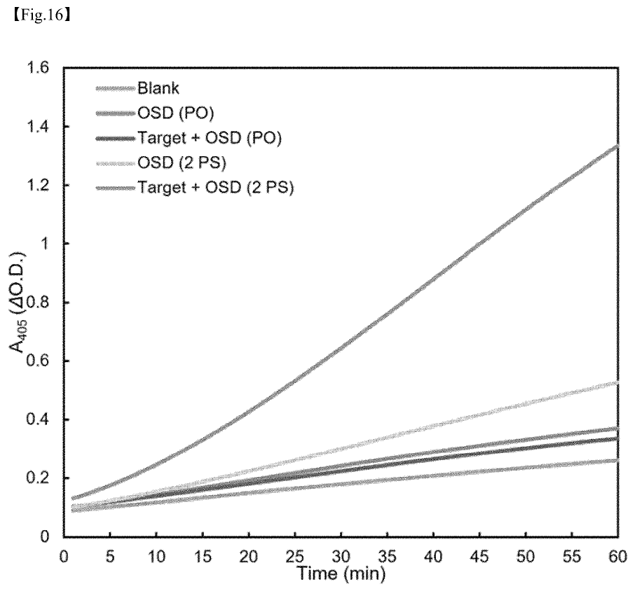

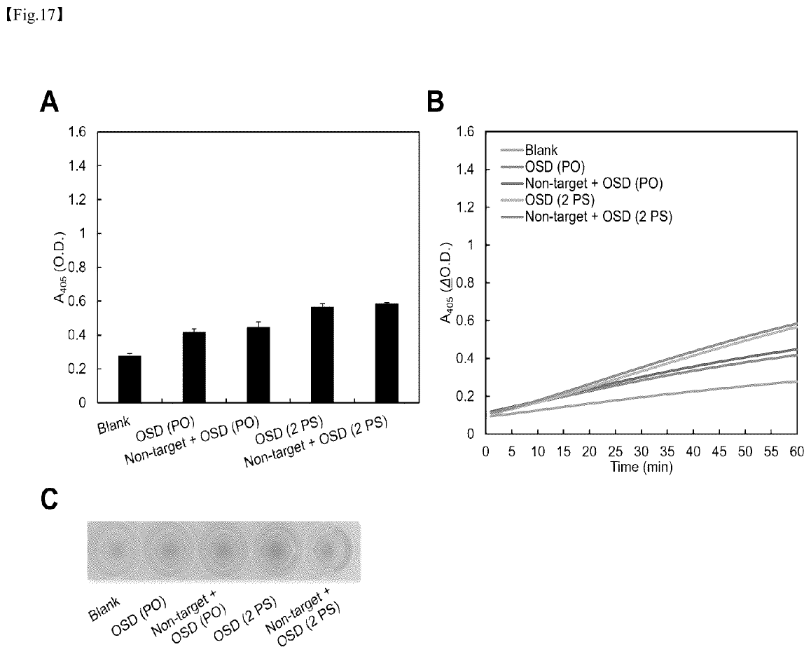

In order to mitigate the possibility of signal leakage through intermolecular DNA hybridization and to lower the instability of the overall intermolecular hybridization due to the aforementioned DNA end breathing and the effect of PS, GC clamping was added to the ends of a portion forming the DNA duplex to increase the stability of the DNA structure. As a result, a hemi-duplex type probe called OSD (intermolecular DNA hybridization) was designed as a probe for an enzymogenic signaling system. In addition to the GC clamping region, a stem structure having an appropriate length was included to ensure stability even when the number of PS increased. OSD may include a 10 nt toehold region in the 5′ or 3′ end direction. A 20 bp substrate strand and a 20 nt output strand that acted on the enzymatic activity were utilized after going through appropriate DNA hybridization conditions. The probe operated in the same manner as in Example 4, and when the target sequence was present, the output strand containing PS was released through a toehold-mediated strand displacement reaction, and enzymatic activity occurred (FIGS. 14A, 14B, and 16). Conversely, in the absence of the target sequence, enzymatic activity remained inactive. To reduce the possibility of signal leakage due to increased DNA instability of the probe, reaction conditions were further optimized (FIG. 15). In addition, when a non-target sequence was present, the probe did not react to release the output strand containing PS, the structure remained stable, and only background signals were detected (FIG. 17). Through these results, it was confirmed that the OSD composed of intermolecular hybridization according to the present disclosure had excellent usability as a probe by considering factors that increased DNA structural stability, such as GC clamping.

Through the above results, the present inventors were able to determine that when sulfur atoms were introduced into nucleic acids using phosphorothioate linkage (PS) modification, the enzymatic activity may be significantly improved, which indicated that the electron density within the nucleobase modified by PS promotes the acceleration of the enzymatic activity through interaction with hemin.

Therefore, since a novel probe for colorimetric detection developed in the present disclosure, in which sulfur atoms were introduced into nucleic acids, may increase enzymatic activity without sequence-dependent limitation, an enzymogenic signaling system using the probe may be usefully applied for sequence-specific colorimetric detection using H-OSD.

Hereinabove, the present disclosure has been described with reference to preferred embodiments thereof. It will be understood to those skilled in the art that the present disclosure may be implemented as modified forms without departing from essential characteristics of the present disclosure. Therefore, the disclosed embodiments should be considered from an illustrative viewpoint rather than a restrictive viewpoint. The scope of the present disclosure is defined by the appended claims rather than by the foregoing description, and all differences within the scope of equivalents thereof should be construed as being included in the present disclosure.

1. A sulfur-containing probe for colorimetric detection, wherein
the probe has the form of a DNA oligonucleotide in which nucleobases are continuously connected, and
the continuous connection of nucleobases is characterized in that any one or more selected from adenine (A), guanine (G), cytosine (C), and thymine (T) are randomly connected, and
a nucleotide phosphate backbone of the DNA oligonucleotide is modified with one or more phosphorothioates so as to include one or more sulfur atoms.

2. The sulfur-containing probe for colorimetric detection of claim 1, wherein the sulfur-containing probe for colorimetric detection has the form of a DNA oligonucleotide in which 10 to 30 nucleobases are continuously connected.

3. The sulfur-containing probe for colorimetric detection of claim 1, wherein the sulfur-containing probe for colorimetric detection enhances enzymatic activity mediated by a colorimetric reaction.

4. The sulfur-containing probe for colorimetric detection of claim 1, wherein when a phosphate backbone of the DNA oligonucleotide is modified with phosphorothioates, at least one of the nucleobases present on either side of the modification is adenine (A).

5. The sulfur-containing probe for colorimetric detection of claim 1, wherein the DNA oligonucleotide including one or more sulfur atoms is a single-stranded DNA oligonucleotide.

6. A composition for colorimetric detection comprising the sulfur-containing probe for colorimetric detection of claim 1.

7. The composition for colorimetric detection of claim 6, further comprising a colorimetric reagent.

8. The composition for colorimetric detection of claim 7, wherein the colorimetric reagent comprises:
at least one selected from the group consisting of 2,2′-azino-bis(3-ethylbenzothiazoline-6-sulfonic acid) (ABTS), o-phenylenediamine dihydrochloride (OPD), diaminobenzidine (DAB), 3-amino-9-ethylcarbazole (AEC), 3,3′,5,5′-tetramethylbenzidine (TMB), AmplexRed, and Homovanilic acid; and
at least one peroxide.

9. The composition for colorimetric detection of claim 6, further comprising hemin.

10. A kit for colorimetric detection comprising the composition for colorimetric detection of claim 6.

11. A colorimetric detection method using the sulfur-containing probe for colorimetric detection of claim 1.

12. A method for preparing a sulfur-containing probe for colorimetric detection, having an effect of enhancing enzymatic activity mediated by a colorimetric reaction, the method comprising:
selecting a target gene to be detected;
designing an oligonucleotide sequence complementary to a nucleic acid sequence of the target gene; and
modifying a nucleotide phosphate backbone in the complementary oligonucleotide with one or more phosphorothioates,
wherein the complementary oligonucleotide has a length of 10 to 30 mer, and the phosphorothioate modification is performed in a region in which at least one of nucleobases present on either side of the modification is adenine (A).
